(19) United States
(12) Patent Application Publication
(10) Pub. No.: US 2017/0239171 A1
(43) Pub. Date: Aug. 24, 2017

(54) OROMUCOSAL FILM PREPARATION

(71) Applicant: Sapiotec GmbH, Wurzburg (DE)

(72) Inventors: Norbert Roewer, Wurzburg (DE); Jens Broscheit, Wurzburg (DE)

(21) Appl. No.: 15/518,990

(22) PCT Filed: Sep. 22, 2015
(86) PCT No.: PCT/EP2015/071644

§ 371 Date: Apr. 13, 2017

(30) Foreign Application Priority Data
Oct. 14, 2014 (EP) 14188811.5

(51) Int. Cl. A61K 9/00; B29C 43/00; B29C 47/00

(52) U.S. Cl. CPC A61K 9/006; B29C 47/0004; B29C 43/003; B29K 2995/0062; B29L 2031/753

(57) ABSTRACT

The Invention relates to an oromucosal film preparation. According to the invention, said oromucosal film preparation comprises an active-ingredient containing surface area (3) and an active-ingredient free surface area (4). Said surface area (4) which is free of active-ingredients can be used as an application aid for the safe and simple application of the film preparation.

The invention relates to an oromucosal film preparation.

The peroral administration of medicaments is widespread. For example, tablets or capsules, which are generally swallowed with liquid, are known.

Likewise known, for example from WO 2012/055947 A1, are active-ingredient-containing thin films, which are placed in the oral cavity, dissolve or disintegrate therein and release the contained active ingredient. The advantage of this administration form is administration without additional liquid and without the burden of swallowing or only with a small burden of swallowing.

Such oromucosal film preparations may be advantageous especially for patient groups who, for example because of their age (children or elderly patients), because of swallowing difficulties or for example because of mental illnesses, exhibit a poor compliance in the intake of conventional oral administration forms.

It is an object of the present invention to provide an oromucosal film preparation of the type mentioned at the start, the use or administration of which is further improved.

This object is achieved by an oromucosal film preparation comprising an active-ingredient-containing plane region and an active-ingredient-free plane region.

First of all, some terms used for the purposes of the invention should be explained.

An oromucosal film preparation (also called orodispersible film or melt film) comprises a combination of at least one carrier (film former) and at least one active ingredient. This combination is shaped to give a thin film, which is placed in the oral cavity, dissolves or disintegrates therein under the influence of moisture and releases contained active ingredients. The thickness of such a film is by preference below 1 mm, more preferably below 500 μm, typically between 100 and 200 μm. These data are the thickness in the dry state.

The active-ingredient-containing plane region is a region which contains one or more active ingredients in a customary and prior-art-known manner and can release said active ingredients in the oral cavity. According to the invention, it is envisaged that the film preparation additionally comprises an active-ingredient-free plane region. In this connection, active-ingredient-free means that said plane region does not comprise any active ingredients or, at most, low amounts of active ingredients.

The purpose of said active-ingredient-free plane region is to facilitate the handling of the film preparation according to the invention. During insertion into the oral cavity, the patient or a third-party support person can grasp the film preparation on the active-ingredient-free plane region and administer it. Thus, it is avoided that the patient or the support person already comes into contact with active ingredient during insertion into the oral cavity. It is especially avoided that active ingredient is already prematurely released from the film preparation, for example owing to skin moisture on the fingers.

By preference, the active-ingredient-free plane region occupies a proportion of 10 to 50%, preferably 10 to 40%, more preferably 20 to 30%, of the total area of the film preparation. In this way, the active-ingredient-free plane region constitutes a sufficiently large handle; apart from that, it is ensured that active ingredient can be released from a sufficiently large active-ingredient-containing plane region. By preference, the total area of the preparation according to the invention can be between 1 and 10 cm².

The active-ingredient-containing plane region and the active-ingredient-free plane region can consist of two different films joined to one another. In this connection, different means that the two plane regions differ at least in terms of their active-ingredient content. The act of joining together can be effected in such a way that a solid connection is present between the two film regions, which connection does not dissolve during normal handling and administration of the film preparation. In this variant of the invention, the active-ingredient-free plane region is generally inserted concomitantly into the oral cavity and disintegrates or dissolves therein, as does the active-ingredient-containing plane region.

Alternatively, it is possible according to the invention to design the connection such that, during the administration of the film preparation, the active-ingredient-free plane region can be separated, preferably torn off, after insertion into the oral cavity. For example, said connection can dissolve rapidly owing to the moisture in the oral cavity, and so, after insertion, the active-ingredient-free plane region becomes detached in a rapid manner and can be removed. It is likewise conceivable for the active-ingredient-free plane region not to be inserted into the oral cavity or to be only partly inserted into the oral cavity, and so said oral cavity cannot be contaminated by the fingers of the person doing the handling.

Film preparations according to the invention comprise film formers. This concerns substances which, as carrier substances, can incorporate active ingredients and which influence or determine the structure, shape and dissolution behavior of the film preparations. In two variants of the invention, the film formers of the two different films or plane regions are identical or different.

In general, identical film formers facilitate production and ensure that the non-active-ingredient-containing plane region, if it remains in the oral cavity after administration, generally behaves similarly, in particular dissolves similarly, to the active-ingredient-containing plane region.

Different film formers may be advantageous when it is desirable to specifically impart different properties to the non-active-ingredient-containing plane region. For example, it may be desirable that said non-active-ingredient-containing plane region dissolves more rapidly in the oral cavity, whereas the active-ingredient-containing plane region remains in the oral cavity for a longer period and releases active ingredient therein.

Alternatively, the non-active-ingredient-containing plane region can be made from a polymer material which is less soluble or possibly not soluble in the environment of the oral cavity, when it is intended that said region be separated from the active-ingredient-containing plane region in the course of administration and not remain in the oral cavity. In this case, the material of the active-ingredient-free plane region can be selected such that, for example, the separation thereof in the course of administration is facilitated.

Film formers suitable according to the invention are absolutely known from the prior art and familiar to a person skilled in the art. For example, the film formers can be selected from the group consisting of cellulose, cellulose derivatives, acrylic and methacrylic acid polymers, polyvinyl alcohols, polyvinyl acetate, polyvinylpyrrolidone and derivatives, polysaccharides and polymers based on starch.

Film formers which may be mentioned by way of example are hydroxyethylcellulose, hydroxypropylcellulose, hydroxypropylmethylcellulose, methylcellulose, Natrosol®, Tylose® H300, Klucel®, Klucel® E, Klucel® L, Klucel® J, Klucel® G, Klucel® M, Klucel® H, Klucel® EF, Klucel® LF, Klucel® JF, Klucel® GF, Klucel® MF, Klucel® HF, Klucel® EXF, Klucel® LXF, Klucel® JXF, Klucel® GXF, Klucel® MXF, Klucel® HXF, Pharmacoat® 603, Pharmacoat® 606, Methocel® E4M, polyethylene glycol-polyvinyl alcohol copolymers, Kollicoat® IR, Kollicoat® protect, sodium carboxymethylcellulose, polysaccharides of maltotriose, Pullulan®, Lycoat® RS720, Lycoat® RS780, Lycoat® NG, and hydroxyethylmethylcellulose.

By preference, the active-ingredient-containing plane region and the active-ingredient-free plane region are distinguishable for the user, by preference optically distinguishable. The film preparation can thus be specifically seized on the active-ingredient-free plane region. Distinguishability can, for example, be brought about by different colors, optical densities, markings, structures or combinations thereof.

The invention further provides a method for producing a film preparation according to the invention. According to the invention, an active-ingredient-free film is joined to an active-ingredient-containing film. The advantage of this method is that an active-ingredient-containing film can be produced in a customary manner and is, only after completion, joined together with and thus combined with an active-ingredient-free, likewise separately produced film as administration aid.

According to the invention, joining can, by preference, be effected thermally, by means of an adhesive or by means of moisture.

If one film or both films have thermoplastic properties, they can, for example, be heated and joined together in the heated state. The thermoplastic properties give rise to a connection sufficiently solid for the purposes of administration.

As adhesive, it is possible to use suitable glues or, for example, viscous polymer solutions, from which solvent is removed by drying and which thereby solidify sufficiently in order to act as adhesive.

If film formers can be dissolved or partly dissolved by suitable solvents, especially water, joining can also be effected under the action of moisture.

In an alternative embodiment of the invention, the active-ingredient-free plane region is joined to the active-ingredient-containing film by film formation in situ. For example, it can be cast onto the finished active-ingredient-containing film or joined by extrusion or pressing.

An exemplary embodiment of the invention will be described schematically on the basis of the drawing, showing:

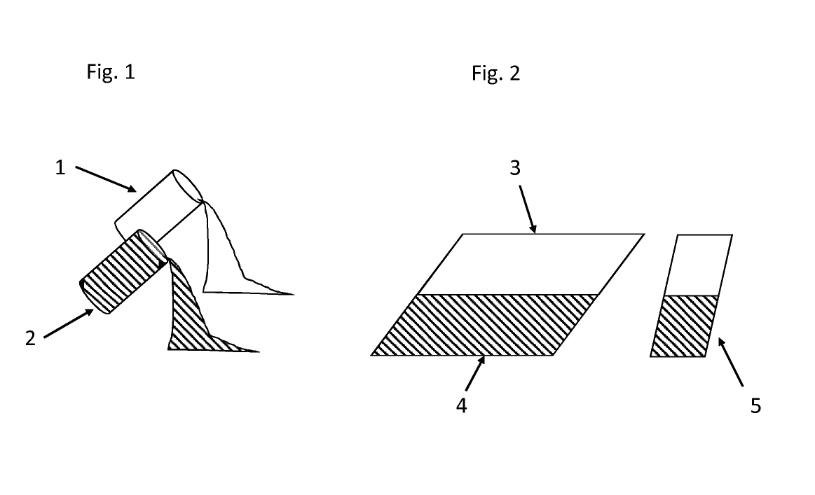

FIG. 1: the production of an inventive film by solvent casting, in schematic form;

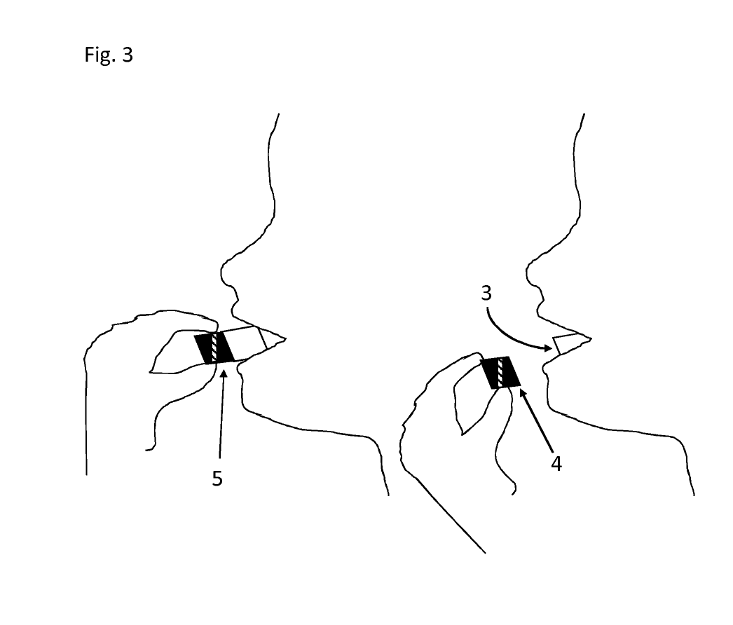

FIG. 2: a view of an inventive film;

FIG. 3: the oral administration of an inventive film.

Suitable example recipes for polymer solutions for producing a film according to the invention are as follows:

Base solution for the active-ingredient-containing film:

| | |
|---|---|
| Hydroxypropylmethylcellulose (Pharmacoat ® 606) | 15% |
| Glycerol, anhydrous | 3% |
| Kollidon ® CL-M | 5% |
| Water | 77% |

Kollidon® CL-M (BASF) is a so-called disintegrant which allows a film to disintegrate in an aqueous environment owing to water uptake and swelling.

Base solution for the active-ingredient-free film:

| | |
|---|---|
| Kollicoat ® IR Carmine | 25% |
| Glycerol, anhydrous | 1% |
| Water | 74% |

FIG. 1 shows schematically how an active-ingredient-free Polymer Solution 1 and an active-ingredient-containing Polymer Solution 2 are jointly cast (solvent casting). The two polymer solutions or layers can be drawn out in a planar manner either in parallel or in succession and adhere to one another. Thereafter, they are dried. The film preparation thus produced comprises an active-ingredient-containing region 3 and an active-ingredient-free region 4.

A preparation produced in this way can be cut into strips of suitable size, labeled altogether by 5, and packaged.

FIG. 3 shows schematically the use. The patient removes the film from the packaging at the active-ingredient-free region 4 and takes the active-ingredient-containing region 3 into the mouth. The hydrophilic polymer of the film begins to dissolve owing to contact with the saliva, and so the active-ingredient-free region 4 can be detached from the active-ingredient-containing region 3 and can be disposed of. Alternatively, it is likewise possible to take the entire film preparation including the active-ingredient-free region 4 into the mouth.

1. An oromucosal film preparation, characterized in that it comprises an active-ingredient-containing plane region (3) and an active-ingredient-free plane region (4).

2. The film preparation as claimed in claim 1, characterized in that the active-ingredient-free plane region (4) occupies a proportion of 10 to 50%, preferably 10 to 40%, more preferably 20 to 30%, of the total area of the film preparation.

3. The film preparation as claimed in claim 1 or 2, characterized in that its total area is 1 to 10 cm².

4. The film preparation as claimed in any of claims 1 to 3, characterized in that the active-ingredient-containing plane region (3) and the active-ingredient-free plane region (4) consist of two different films joined to one another.

5. The film preparation as claimed in claim 4, characterized in that the film formers of the two different films are identical.

6. The film preparation as claimed in claim 4, characterized in that the film formers of the two different films differ.

7. The film preparation as claimed in any of claims 1 to 6, characterized in that the film formers are selected from the group consisting of cellulose, cellulose derivatives, acrylic and methacrylic acid polymers, polyvinyl alcohols, polyvinyl acetate, polyvinylpyrrolidone and derivatives, polysaccharides and polymers based on starch.

8. The film preparation as claimed in any of claims 1 to 7, characterized in that the active-ingredient-containing plane region (3) and the active-ingredient-free plane region (4) are optically distinguishable.

9. A method for producing a film preparation as claimed in any of claims 1 to 7, characterized in that an active-ingredient-free film is joined to an active-ingredient-containing film.

10. The method as claimed in claim 8, characterized in that joining is effected thermally, by means of an adhesive or by means of moisture.

11. The method as claimed in claim 8, characterized in that the active-ingredient-free plane region (4) is joined to the active-ingredient-containing film by film formation in situ.

12. The method as claimed in claim 10, characterized in that the film formation in situ comprises extrusion and/or pressing.
